(19) United States
(12) Patent Application Publication
(10) Pub. No.: US 2009/0252815 A1
(43) Pub. Date: Oct. 8, 2009

(54) Pulverulent mixtures containing hydrogen peroxide and hydrophobized silicon dioxide

(75) Inventors: Egon Walzer, Maintal (DE); Corinna Schmidt, Steinau (DE); Friedhelm Brandner, Erlenbach (DE); Steffen Hasenzahl, Morris Plains, NJ (US); Mario Scholz, Grundau (DE); Birgit Del Grosso, Freigericht (DE); Heike Riedmann, Mobris (DE)

Correspondence Address: LAW OFFICE OF MICHAEL A. SANZO, LLC, 15400 CALHOUN DR., SUITE 125, ROCKVILLE, MD, 20855, US

(73) Assignee: Evonik Degussa GmbH, Essen (DE)

(21) Appl. No.: 12/320,349

(22) Filed: Jan. 23, 2009

Related U.S. Application Data
(63) Continuation-in-part of application No. 11/284,399, filed on Nov. 18, 2005, now abandoned.
Application No. 11/284,399 is a continuation of application No. PCT/EP04/04954, filed on May 10, 2004.
Application No. PCT/EP04/04954 is a continuation-in-part of application No. 10/558,263, filed on Nov. 21, 2005, now abandoned.

(30) Foreign Application Priority Data
May 23, 2003 (DE) DE 103 23 840.9
Jan. 15, 2004 (DE) DE102004002 356.5
Jan. 15, 2004 (DE) DE102004002355.7

(51) Int. Cl. A61K 33/40; A61P 31/00; A61P 17/02; A61P 17/10

(52) U.S. Cl. 424/616

(57) ABSTRACT

The present invention is directed to pulverulent mixtures comprising hydrogen peroxide and hydrophobized, pyrogenically prepared silicon dioxide powder, preferably with a methanol wettability of at least 40. The pulverulent mixtures exhibit good storage stability and can be used for the controlled release of hydrogen peroxide and/or oxygen. The invention also includes methods of making these pulverulent mixtures and methods of using the mixtures in detergents, cleaning compositions, topical medications, antimicrobials and other products.

### CROSS REFERENCE TO RELATED APPLICATIONS

The present application is a continuation-in-part of U.S. application Ser. No. 11/284,399, filed on Nov. 18, 2005. U.S. Ser. No. 11/284,399 is a continuation of international application PCT/EP2004/004954, which has an international filing date of May 10, 2004, and which was published in German under PCT Article 21(2) on Dec. 2, 2004. PCT/EP2004/004954 claims priority to German applications DE 103 23 840.9, filed on May 23, 2003, and DE 10 2004 002 356.5, filed on Jan. 15, 2004. The present application is also a continuation-in-part of U.S. application Ser. No. 10/558,263, filed in the US on Nov. 21, 2005. U.S. Ser. No. 10/558,263 is US national stage of international application PCT/EP04/005220 which has an international filing date of May 14, 2004, and which was published in English under PCT Article 21(2) on Dec. 2, 2004. PCT/EP04/005220 claims priority to German applications DE 103 23 840.9, filed on May 23, 2003 and DE 10 2004 002 355.7, filed on Jan. 15, 2004. All of these prior applications are incorporated herein by reference in their entirety.

### FIELD OF THE INVENTION

The present invention is directed to pulverulent mixtures containing hydrogen peroxide and hydrophobized silicon dioxide for the controlled release of hydrogen peroxide and/or oxygen. The invention also includes methods for the preparation and use of these mixtures.

### BACKGROUND OF THE INVENTION

Hydrogen peroxide is produced and marketed in the form of aqueous solutions. However, for many applications, it is advantageous to use a solid storage form of hydrogen peroxide instead of the aqueous solutions. Commercial solid storage forms for hydrogen peroxide are sodium carbonate perhydrate, sodium perborate and the adduct of urea with hydrogen peroxide. A disadvantage of these storage forms, however, is that in addition to hydrogen peroxide, they introduce other water-soluble constituents which are undesirable in many applications, for example because in aqueous solutions they lead to changes in pH, increase salt content or increase the content of organic substances.

Pulverulent products which contain aqueous hydrogen peroxide, at least 9 wt % of a finely dispersed hydrophobized silicon dioxide and which do not display the aforementioned disadvantage are known from German laid-open specification DE 20 137 63. As applications of these products, DE 20 137 63 cites bleaching, in particular of products containing oils, fats and cellulose, and addition to cleaning agents and cosmetic products. Apart from the property that dry powders are obtained with good stability, no other application-related properties are cited in DE 20 137 63. To ensure an adequate flowability of the mixture, the content of hydrophobized silicon dioxide should preferably be between 10 and 35 wt %. Hydrophobic silicon dioxides which are employed are those which have been hydrophobized with dimethyldichlorosilane or fatty alcohols having 8 to 26 carbon atoms.

One disadvantage of these mixtures is the limited stability of hydrogen-peroxide in mixture with hydrophobic silicon dioxide. Although this is described as good in DE-A-2013763 and can be increased further by known stabilizing agents for hydrogen peroxide, for many uses, such as, for example, as an additive to cleaning compositions, the necessary long-term stability does not exist. The relatively high content of hydrophobic silicon dioxide is a further disadvantage. In addition, in many applications in which hydrogen peroxide is used in the form of a solid storage form, it is also desirable for hydrogen peroxide to be released from the solid storage form in a controlled manner, e.g., by means of a delayed release over an extended period of time or a release in response to a changed physical variable.

### DESCRIPTION OF THE INVENTION

The invention is directed to mixtures of silicon dioxide powder and hydrogen peroxide which have long-term stability. It has also been found that, through the use of pulverulent mixtures containing hydrogen peroxide and hydrophobized silicon dioxide, a controlled release of hydrogen peroxide can be achieved in a simple manner without the release of other water-soluble substances. Molecular oxygen can be released instead of, or together with, hydrogen peroxide.

Pulverulent Mixtures

In a first aspect, the invention is directed to storage-stable pulverulent mixtures comprising hydrogen peroxide and hydrophobized, pyrogenically prepared silicon dioxide powder. Based on the total weight of the mixture, the hydrogen peroxide should be present at between 5 and 70 wt % and preferably at between 10 and 50 wt %. The hydrophobized silicon dioxide powder should have a methanol wettability of at least 40 and be present in the mixtures at less than 9 wt %, based on the total weight of the mixture.

Methanol wettability is a measure of the hydrophobicity of the silicon dioxide and is defined as the methanol content of a methanol-water mixture in percent by volume at which 50% of the hydrophobized silicon dioxide introduced into the methanol-water mixture forms a sediment. With a lower methanol content, wetting does not take place and most of the hydrophobized silicon dioxide floats. With a higher methanol content, extensive wetting takes place and the majority of the silicon dioxide forms a sediment. Through the use of pyrogenically produced, hydrophobized silicon dioxide having a methanol wettability of at least 40, pulverulent mixtures having a particularly good storage capacity are obtained which are in the form of free-flowing powders, even with hydrophobized silicon dioxide contents of less than 9 wt. %.

The invention also encompasses processes for the preparation of the pulverulent mixtures in which hydrogen peroxide is present in the form of drops of an aqueous solution which are enclosed by hydrophobized silicon dioxide. Such pulverulent mixtures can be produced by the intensive mixing of an aqueous hydrogen peroxide solution with hydrophobized silicon dioxide. Any mixing unit which can deliver sufficient energy to ensure a rapid division of the liquid into small droplets, which are then immediately surrounded by hydrophobized silicon dioxide powder, is suitable for this purpose.

The silicon dioxide used in the processes should preferably have a methanol wettability of at least 40 and should be mixed with the aqueous hydrogen peroxide at a temperature of not more than 70° C. Any compound that can be attached to the silicon dixoide particles and produce the desired wettability can be used for hydrophobization. Preferred hydrophobizing compounds include: octamethyloyclotetrasiloxane, polydimethylsiloxane, octylsilane and/or hexamethyldisilazane and have a specific surface area of between 10 and 400, and more preferably between 80 and 300 m²/g. Examples of hydrophobized silicon dioxide powders that be used to make pulverulent mixtures are shown in table 1.

TABLE 1
Hydxophobized silicon dioxide powders suitable for the preparation
of the powder mixture according to the invention

| | | Spec. | |
|---|---|---|---|
| | | surface area | |
| Hydrophobized | Hydrophobizing | (approx.) | Methanol |
| SiO₂ | agent | m²/g | wettability |
| Aerosil ® | Octamethylcyclotetrasiloxane | 150 | 40 |
| R104 | | | |
| Aerosil ® | Octamethylcyclotetrasiloxane | 250 | 45 |
| R106 | | | |
| Aerosil ® | Polydimethylsiloxane | 100 | 70 |
| R202 | | | |
| Aerosil ® | Octylsilane | 150 | 45 |
| R805 | | | |
| Aerosil ® | Hexamethyldisilazane | 260 | 50 |
| R812 | | | |
| Aerosil ® | Hexamethyldisilazane | 220 | 60 |
| R812S | | | |
| Aerosil ® | Hexamethyldisilazane | 150 | 65 |
| R8200 | | | |

Hydrogen peroxide should, preferably, be present in the aqueous solutions at between 5 and 70 wt % and more preferably at between 10 and 50 wt %. These solutions may be stabilized against decomposition to water and oxygen using stabilizers. The type and amount of stabiliser added will influence whether it is predominantly hydrogen peroxide or predominantly oxygen which is released when the pulverulent mixtures are used. Suitable stabilizers are stannates, phosphates, pyrophosphates, nitrates, magnesium salts, phosphonic acid, aminophosphonic acids, EDTA, gelatine and mixtures thereof, which are added in amounts of between 0.01 and 1 wt %. It is also possible to employ solutions which are not stabilized for the preparation of the powder mixtures. However, in this case, a lower stability of the powder must be expected.

Uses of Pulverulent Mixtures

The invention provides for the use of the mixtures described above in detergents, in cleaning compositions and in hair and skin treatment compositions. In one embodiment, the powder mixtures according to the invention can be used as a bleach component in detergents together with or, preferably, in the place of inorganic peroxygen compounds such as sodium perborate or sodium carbonate perhydrate.

The powder mixtures according to the invention can also be used as bleaching or antiseptic components in other cleaning compositions. In contrast to inorganic peroxygen compounds, the powder mixtures according to the invention provide a bleaching or antiseptic action without the addition of water. Thus, the mixtures will be especially useful in compositions such as stain removers for textiles, upholstery, carpets and carpet flooring which are used without the addition of water, e.g., in powder form.

In addition, the powder mixtures according to the invention can be used as a bleach component in hair treatment compositions, preferably in amounts of 20 to 80 wt %. The hair-bleaching compositions should generally include at least one alkaline component (preferably chosen from hydroxides, carbonates, hydrogen carbonates and silicates of alkali metals or alkaline earth metals) in an amount of 10 to 40 wt %. The hair-bleaching compositions preferably also comprise one or more surfactants, with both nonionic and anionic, cationic or zwitter-ionic surfactants being suitable. In addition, these compositions may include auxiliary substances, such as, for example, nonionic, anionic or cationic polymers, thickeners, protein hydrolysates, phospholipids, metal complexing agents, dyestuffs and perfume oils. Corresponding hair-bleaching compositions are known in the art, for example from WO 01/45658, with peroxodisulfates or other inorganic peroxides as a bleach component. In embodiments of the present invention, the peroxodisulfates are replaced completely or in part by the powder mixture described herein.

The powder mixtures according to the invention can additionally be used as oxidizing agents in hair treatment compositions for the permanent coloring of hair with oxidation dyestuffs. In this case, shortly before use of the hair-coloring composition, the powder mixtures are mixed with a formulation which comprises the precursors of a developer component and coupling component for the oxidation dyestuff. The powder mixtures can be mixed directly with the formulation of the dyestuff precursors or dispersed in an aqueous solution or emulsion beforehand. The dyestuff precursors are preferably formulated as aqueous emulsions which also comprise, in addition to a developer component and coupling component, one or more emulsifiers as well as one or more liquid non-polar components and, optionally, further auxiliary substances. Corresponding formulations of dyestuff precursors are known in the art, for example from DE 199 01 886, for use with liquid hydrogen peroxide formulations. In embodiments of the present invention, the liquid hydrogen peroxide formulations are replaced completely or in part by the powder mixtures described herein.

The powder mixtures according to the invention can also be used as oxidizing agents in skin treatment compositions for cosmetic purposes, such as, for example, for brightening skin or for removing pigmental moles and freckles. Corresponding skin treatment compositions are known in the art, with inorganic peroxides, such as, for example, zinc peroxide or urea peroxide, organic peroxides, hydroquinone or basic bismuth salts as active compounds. In embodiments of the present invention, the powder mixtures described herein are used instead of these active compounds or in addition to one or more of these active compounds.

In another aspect, the powder mixtures according to the invention can be used for the preparation of compositions for disinfection of the skin and compositions for the treatment of acne or psoriasis. Corresponding compositions for treating acne or psoriasis are known in the art and utilize organic peroxides, such as, for example, benzoyl peroxide, as active compounds. In embodiments of the present invention, the powder mixtures described herein are used instead of the organic peroxides.

Finally, the powder mixtures according to the invention can be used as hardeners for curing formulations by means of free radicals. Examples of such formulations include resins, lacquers and adhesives based on vinyl ester resins, unsaturated polyester resins or crosslinkable silicones. Corresponding formulations are known in the art and use organic peroxides for curing. In embodiments of the present invention, the powder mixtures described herein are used instead of the organic peroxides. This has the advantage that the properties of the cured products are not adversely influenced by cleavage products of the organic peroxides which may cause odor or result in discoloration of the product.

Controlled Release Compositions

In preferred embodiments of the invention, the pulverulent mixtures containing hydrogen peroxide and hydrophobized silicon dioxide are used for the time-delayed release of hydrogen peroxide, oxygen or both hydrogen peroxide and oxygen. Most preferably, the mixtures are used for the time-delayed release of hydrogen peroxide into an aqueous medium. To this end, the pulverulent mixtures are brought into contact with the aqueous medium and optionally dispersed therein, dispersion preferably being performed at low shear forces. In a preferred embodiment, the pulverulent mixture is brought into contact with the aqueous medium in a container which is permeable to water and hydrogen peroxide but impermeable to the hydrophobized silicon dioxide.

The containers used for this purpose preferably consist entirely, or partly, of a filter medium whose pore size is smaller than the average size of the particles of hydrophobized silicon dioxide in the pulverulent mixtures. In one embodiment of the invention, the container with the pulverulent mixture is immersed in the aqueous medium such that transport of hydrogen peroxide out of the container and into the medium takes place largely by means of diffusion. In another embodiment, the aqueous medium flows through the container. The containers can be of any shape and can take the form of a filter bag, a filter candle or a cartridge. Through the use of such containers, hydrogen peroxide can be released in a time-delayed manner into an aqueous medium and, at the same time, the hydrophobized silicon dioxide is retained in the container.

The timed release pulverulent mixtures described above can be used to maintain defined concentrations of hydrogen peroxide in an aqueous medium for extended periods without severe fluctuations in concentration and without the need for complex metering and regulating equipment. This may be particularly advantageous, for example, in the release of hydrogen peroxide into aquariums and into containers or ponds used for rearing fish. In addition, the timed release pulverulent mixtures may be used to maintain a hydrogen peroxide concentration sufficient to prevent the multiplication of micro-organisms but low enough to be safe for fish and fish larvae without the need for regulating equipment.

In another embodiment, the pulverulent mixtures containing hydrogen peroxide and hydrophobized silicon dioxide are used to release hydrogen peroxide in response to the application of pressure. This pressure can be applied mechanically (e.g., by means of a plunger), hydraulically by means of liquid pressure, or pneumatically by means of gas pressure. The application of mechanical or pneumatic pressure results in hydrogen peroxide being released in the form of an aqueous solution which has substantially the same concentration as the hydrogen peroxide solution used to produce the pulverulent mixture.

Preferably, hydraulic pressure is used to release hydrogen peroxide into an aqueous medium. In this embodiment, the pulverulent mixture is preferably placed in a container which is permeable to water and hydrogen peroxide but impermeable to the hydrophobized silicon dioxide. The aqueous medium then flows through the container, with pressure resulting from the dynamic flow of the aqueous medium. In this way, hydrogen peroxide is released at a controlled rate without the need for metering or regulating equipment.

The pulverulent mixtures containing hydrogen peroxide and hydrophobized silicon dioxide can also be used for the controlled release of hydrogen peroxide into a pulverulent medium. Preferably the hydrogen peroxide and silicon dioxide mixtures represent between 0.1 and 10 wt % of the total weight of the of the compositions and hydrogen peroxide is released into the pulverulent medium in a time-delayed manner so as to maintain a low concentration over an extended period of time. The free-flowing properties of the pulverulent medium can be improved through the addition of the mixtures containing hydrogen peroxide and hydrophobized silicon dioxide and, in addition, pressure can be applied to induce the release of hydrogen peroxide at a defined time. The release of hydrogen peroxide through the application of pressure can be used, inter alia, to start a chemical reaction in the pulverulent medium, e.g., to cure the medium.

The pulverulent mixtures containing hydrogen peroxide and hydrophobized silicon dioxide can also be added to emulsions, gels, creams or pastes for the controlled release of hydrogen peroxide and/or oxygen in the preparations thus obtained. The mixtures may be used to maintain a low concentration of hydrogen peroxide in the preparations for an extended period of time. Alternatively, the pulverulent mixtures can be used to release hydrogen peroxide into the preparation at a defined time by the application of pressure or by the application of shear forces. For example, in a preferred embodiment, the pulverulent mixtures are used in cosmetic preparations to release hydrogen peroxide in response to pressure produced by massaging compositions into the skin. The release of hydrogen peroxide through the application of pressure or the application of shear forces can also be used to trigger a chemical reaction in preparations and to thereby alter its properties. Such preparations can, for example, take the form of adhesives which crosslink and cure under the application of pressure.

The pulverulent mixtures containing hydrogen peroxide and hydrophobized silicon dioxide can also be used for the controlled release of hydrogen peroxide into an aqueous medium, a pulverulent medium, an emulsion, a gel, a cream or a paste, for the purpose of inhibiting the growth of microorganisms or, depending on the amount of hydrogen peroxide released, for the purpose of killing microorganisms. Thus, the time-delayed release of hydrogen peroxide can be used to preserve the medium into which it is released.

Compositions that respond to pressure can be used to deliberately release hydrogen peroxide at a particular moment in a quantity effective to destroy microorganisms and thereby provide a disinfectant action. Compositions of this type will be useful as wound treatment agent and may take the form of plasters, creams, ointments or gels.

In another embodiment, the pulverulent mixtures containing hydrogen peroxide and hydrophobized silicon dioxide can be used for the controlled release of oxygen into a gaseous medium. The release of oxygen preferably takes place in a time-delayed manner such that a specific oxygen concentration is maintained in the gaseous medium over an extended period of time. This allows for a constant level of oxygen even when oxygen is withdrawn, for example due to metabolic reactions of microorganisms. By maintaining an adequate oxygen concentration, the growth of anaerobic microorganisms can be inhibited and hence the formation of odour causing volatile metabolic products by such microorganisms can be reduced or eliminated. This would be particularly advantageous in hygiene products which are worn on the body, in food packaging, in storage containers for rotting waste and in air filters.

### EXAMPLES

### Analytical Methods

Determination of the Methanol Wettability

In each case 0.2 g (±0.005 g) of hydrophobic silicon dioxide powder is weighed into transparent centrifuge tubes. 8.0 ml. of a methanol/water mixture with, in each case, 10, 20, 30, 40, 50, 60, 70 and 80 vol % methanol are added to each weighed portion. The closed tubes are shaken for 30 seconds and then centrifuged at 2,500 min⁻¹ for 5 minutes. The sediment volumes are read, converted into percent and plotted against the methanol content (vol %) on a graph. The point of inflection of the curve corresponds to the methanol wettability.

Determination of the Hydrogen Peroxide Content

Hydrogen peroxide is reduced by iron(II) sulfate in sulfuric acid solution. The excess iron(II) sulfate is back-titrated with potassium permanganate solution. The titration is controlled by a Titroprocessor 682 with sample changer from Metrohm.

Procedurally, approximately 0.6500-0.7000 g of a powder mixture of hydrogen peroxide and hydrophobized silicon dioxide is acidified with 25 ml of 25 percent sulfuric acid. 10 ml of an iron(II) sulfate solution (69.5 g/l iron(II) sulfate heptahydrate) are then pipetted in, and thereafter 50 ml of completely demineralized water are added. The mixture is mixed thoroughly with a propeller stirrer for 15 minutes and subsequently titrated with KMnO₄ solution (0.05 molar). The end point of the titration is determined potentiometrically. The consumption of KMnO₄ solution for the amount of iron(II) sulfate solution employed is called the blank value.

Calculation:

$$\frac{( blank value \text{-} consumption ) ml K Mn {O}_{4} \cdot 42.52 \cdot 100}{weight ( g ) \cdot 1 \text{,} 000}$$

### Example 1

### Preparation of Pulverulent Mixtures Containing Hydrogen Peroxide and Hydrophobized Silicon Dioxide

93 g of a 10% hydrogen peroxide solution are mixed with 7 g of Aerosil® R812S in a multimixer (Braun, model MX32) at the highest setting for 45 s. The high shear forces of the mixer reduce the liquid to small droplets, which are enclosed by the hydrophobic Aerosil. The mixture formed is a free-flowing powder.

### Examples 24

### Additional Mixtures According to the Invention

Examples 2 to 4 were carried out analogously to give powders according to the invention.

### Examples 5-7

### Comparative Examples

Examples 5 to 7 are comparison examples. The starting materials and properties of powder mixtures with hydrogen peroxide and hydrophobized silicon dioxide are listed in table 2.

TABLE 2
Starting materials and properties of powders comprising hydrogen
peroxide and hydrophobized silicon dioxide powder

| | | Example | | | | | | |
|---|---|---|---|---|---|---|---|---|
| | | 1 | 2 | 3 | 4 | 5 | 6 | 7 |
| Content of H₂O₂ in the solution | g | 93.0 | 93.0 | 93.0 | 95.0 | 91.0 | 91.0 | 85.0 |
| Conc. of H₂O₂ in the solution | wt. % | 10.0 | 35.0 | 50.0 | 10.0 | 10.0 | 10.0 | 10.0 |
| Hydrophobized | | R812S | R812S | R812S | R202 | R972 | R972 | R816 |
| Aerosil | | | | | | | | |
| Content of hydrophobized | g | 7.0 | 7.0 | 7.0 | 5.0 | 9.0 | 7.0 | 15.0 |
| Aerosil | | | | | | | | |
| Methanol wettability | | 60 | 60 | 60 | 70 | 35 | 35 | 0 |
| Content of H₂O₂ in the powder | | | | | | | | |
| after 0 days | wt. % | 10.1 | 35.1 | 50.1 | 10.07 | 35.1 | n.a. | n.a. |
| after 30 days | | 10.05 | 34.95 | 45.6 | 9.98 | 29.2 | | |
| after 60 days | | 9.5 | 34.8 | 40.5 | 9.37 | 23.8 | | |

* n.a. = no flowable powder;

Examples 1 to 4 show that when a hydrophobized silicon dioxide powder with a methanol wettability of at least 40 is used, free-flowing powders with a high stability are obtained even with very low contents of 7 and 5 wt %. Examples 6 and 7 show that when hydrophobized silicon dioxide powders with a methanol wettability of less than 40 are used, no free-flowing powder is obtained.

In example 5, a free-flowing powder is indeed obtained with a hydrophobized silicon dioxide powder with a methanol wettability of less than 40, but here also a lower stability of the hydrogen peroxide manifests itself.

### Examples 8 and 9

### Use in a Hair Treatment Composition for Bleaching Hair

TABLE 3
Composition of hair-bleaching powders and the associated
hydrogen peroxide developer solution in wt %

| | | |
|---|---|---|
| Constituents | Example 8 | Example 9 |

Hair Bleaching Powder

| | | |
|---|---|---|
| Ammonium peroxodisulfate | — | 30.0 |
| Potassium peroxodisulfate | — | 30.0 |
| Product from example 3 | 38.8 | — |
| Sodium metasilicate | 27.6 | 18.0 |
| Sodium stearate | 15.3 | 10.0 |
| Magnesium carbonate | 12.2 | 8.0 |
| Protein hydrolysate | 1.5 | 1.0 |
| Sodium carboxymethylcellulose | 3.8 | 2.5 |
| Ethylenediaminetetraacetic acid, | 0.8 | 0.5 |
| disodium salt | | |

Developer solution

| | | |
|---|---|---|
| Hydrogen peroxide, 50 wt. % | 24.00 | 24.00 |
| Phosphoric acid, 85 wt. % | 0.50 | 0.50 |
| Acetanilide | 0.01 | 0.01 |
| Water | 75.49 | 75.49 |

The constituents of the hair-bleaching powder were weighed into a 1,000 ml glass vessel in the ratio of amounts shown in table 3 (total batch: 500 g). After the glass vessel had been closed, the components were mixed gently in a free-fall mixer (Turbula, Bachofen) at 42 rpm for 10 min. The developer solution (total amount also 500 g) was prepared by initially introducing water into a cleaned glass vessel and adding hydrogen peroxide, phosphoric acid and acetanilide in the ratios of amount of table 3, while stirring with a glass rod.

The hair-bleaching powder and developer were mixed in a ratio of 1:1. In each case, 2.0 g of this mixture were applied to 0.5 g of hanks of dark blond hair (Fischbach+Miller, code 6923). After an action time of 30 minutes, the mixture was rinsed out of the hanks of hair and the hair was dried in a drying cabinet at 40° C. for 2 h and then evaluated visually. The hair bleached with the mixture from example 8 was significantly lighter than that treated with the mixture from comparison example 9.

### Examples 10 and 11

### Use in a Hair Treatment Composition for Coloring Hair

To prepare the coloring cream, 395.00 g of deionized water and 35.0 g of aqueous 25 wt % ammonia solution were initially introduced into a 3 l Stephan mixer (Stephan UMC 5 electronic, A. Stephan und Sohne GmbH & Co., Hameln, Germany). All further constituents were added and mixed in slowly in the ratios of amounts of table 4. The mixture was then homogenized for 10 min at 1,000 rpm, so that a uniform cream was formed, which was then transferred to a thoroughly cleaned one liter glass bottle with a screw cap. To prepare the developer emulsion, 442.5 g of water were initially introduced into the cleaned Stephan mixer and all further components were added in the ratios of amounts of table 4, with slow stirring. This mixture was then mixed at 50 rpm for 15 min until a homogeneous, slightly viscous mass had formed. This was in turn transferred to a thoroughly cleaned one liter glass bottle with a screw cap.

The coloring cream and developer emulsion were mixed in a ratio of 1:1 directly before use. In each case 2.0 g of this mixture were applied to 0.5 g of hanks of dark blond hair (Fischbach+Miller, code 6923). After an action time of 25 minutes, the mixture was rinsed out and the hair was dried in a drying cabinet at 40° C. for 2 h and then evaluated visually. Both mixtures lead to a light copper shade, with the mixture from example 10 leading to a stronger colour shade. Furthermore, it was significantly more viscous and therefore easier to apply than that from comparison example 11.

TABLE 4
Composition of the colouring cream and the
associated developer emulsion in wt. %

| | | |
|---|---|---|
| | | Example 11 |
| | | (comparison |
| Constituents | Example 10 | example) |

Colouring cream

| | | |
|---|---|---|
| C₁₂-C₁₈-Fatty alcohol mixture | 7.00 | 7.00 |
| Lanolin | 1.50 | 1.50 |
| C₁₂₋₁₈-Fatty alcohols•20 EO | 1.50 | 1.50 |
| Lanolin alcohol•5 EO | 1.00 | 1.00 |
| Cationic polymer | 1.00 | 1.00 |
| Ammonium sulfate | 0.50 | 0.50 |
| Sodium sulfite | 0.50 | 0.50 |
| Ethylenediaminetetraacetic | 0.10 | 0.10 |
| acid, disodium salt | | |
| 2,4-Diaminophenol | 0.30 | 0.30 |
| dihydrochloride | | |
| 5-Amino-2-methylphenol | 0.21 | 0.21 |
| 2,5-Diaminotoluene sulfate | 0.07 | 0.07 |
| Resorcinol | 0.04 | 0.04 |
| Ammonia, 25 wt. % | 7.00 | 7.00 |
| Perfume oil | 0.30 | 0.30 |
| Water | 78.98 | 78.98 |

Developer emulsion

| | | |
|---|---|---|
| Hexadecyl alcohol | 3.00 | 3.00 |
| C_{12/18}-Fatty alcohol•20 EO | 1.00 | 1.00 |
| C_{16/18}-Fatty alcohol sulfate | 1.00 | 1.00 |
| Hydrogen peroxide, 50 wt. % | — | 6.00 |
| Product from example 4 | 6.45 | — |
| Phosphoric acid, 85 wt. % | 0.50 | 0.50 |
| Acetanilide | 0.01 | 0.01 |
| Water | 88.04 | 88.49 |

### Examples 12 and 13

### Use in Skin Treatment Compositions for Cosmetic Treatment of Acne

To prepare the acne gel from example 12, ethanol was initially introduced into a Stephan mixer. The other components were added in the ratio of amounts stated in table 5, with slow-stirring (total batch: 500 g) and the mixture was then homogenized gently at 50 rpm for 15 minutes.

TABLE 5
Composition of a gel-like skin treatment composition in wt. %

| Constituents | Example 12 |
|---|---|
| Product from example 3 | 3.69 |
| Ethanol | 91.31 |
| Veegum | 0.5 |
| Macrogol lauryl ether | 2.5 |
| Hypromellose | 2.0 |

The acne cream of example 13 was prepared by adding to the lipophilic cream base (Deutscher Arzneimittelkodex, Neues Rezeptur-Formularium 1999 [German-Pharmaceuticals Codex, New Recipe Formularium 1999], supplement 16, NRF 11.104, Govi-Verlag, Deutscher Apotheker-Verlag), which had been initially introduced into a Stephan mixer, all further components in the ratios of amounts stated in table 6, with slow stirring (total batch: 500 g) and the resulting mixture was then homogenized gently at 50 rpm for 15 min.

TABLE 6
Composition of a skin treatment composition
in the form of a cream in wt. %

| Constituents | Example 13 |
|---|---|
| Product from example 4 | 7.56 |
| O/W Ointment base | 87.34 |
| Triton X-200 | 1.0 |
| Sodium lauryl sulfoacetate | 2.0 |
| Sodium dihexyl sulfosuccinate | 2.0 |
| Imide-urea | 0.1 |

### Example: 14

### Use in a Powdered Detergent

TABLE 7
Composition of a powdered detergent in wt. %

| Constituents | Example 14 |
|---|---|
| Product from example 2 | 12.0 |
| Linear alkylsulfonate | 10.0 |
| Ethoxylated fatty alcohols | 5.3 |
| Sodium soap | 3.7 |
| Defoamer | 4.4 |
| Zeolite | 32.3 |
| Sodium carbonate | 13.2 |
| Copolymer | 2.7 |
| Sodium silicate | 3.5 |
| Carboxymethylcellulose | 1.5 |
| Phosphonic acid | 3.5 |
| Optical brightener | 0.2 |
| Sodium sulfate | 7.3 |
| Protease | 0.5 |

### Example 15

### Use for Curing an Unsaturated Polyester Resin

1.7 parts by weight of pyrogenic silica (AEROSIL 200) are added to 62 parts by weight of Palatal A 410-01 (unsaturated polyester resin). 18 parts by weight of styrene are then added. Thereafter, 0.5 part by weight of BYK-A 555 are mixed in as a de-aerating additive. Finally, for crosslinking, 1 part by weight of the powder mixture according to the invention from example 2 is added. Thereafter, the mixture cures completely, without discoloration.

### Example 16

### Time-delayed Release of Hydrogen Peroxide into an Aqueous Medium

4 g of the product obtained in Example 1 were introduced into a commercial teabag and the teabag was sealed. The teabag was completely submerged in 1 litre of demineralised water in a stirred vessel and the solution was stirred slowly at room temperature. Samples were taken at regular intervals and the hydrogen peroxide content of the solution determined by cerimetric titration.

### Example 17

### Time-delayed Release of Hydrogen Peroxide into an Aqueous Medium (Additional Examples)

This example was performed in the same way with the product obtained in Example 2.

TABLE 8
Time-delayed release of H₂O₂ into an aqueous medium

H₂O₂ content in g/l

| Time in hr | Example 16 | Example 17 |
|---|---|---|
| 0 | 0 | 0 |
| 2 | 0.02 | 0.08 |
| 4 | 0.03 | 0.17 |
| 6 | 0.06 | 0.52 |
| 16 | 0.08 | 0.77 |
| 24 | 0.10 | 1.03 |
| 48 | 0.17 | 1.08 |
| 72 | 0.23 | 1.17 |

The results summarised in Table 8 show that hydrogen peroxide was released from the teabags into the surrounding aqueous medium in a time-delayed manner over a period of over 48 h.

### Example 18

### Release of Hydrogen Peroxide Through Application of Pressure

10 g of the product obtained in Example 1 were subjected to an overpressure of 4 bar of compressed air in a pressure filter (pore size 1.2 μm). The hydrogen peroxide released under the application of pressure was collected in a reservoir. 33% of the hydrogen peroxide contained in the product had been collected in the form of an aqueous solution within 15 min and a total of 50% of the initial amount within 30 min.

### Example 19

### Release of Hydrogen Peroxide Through Application of Pressure

10 g of the product obtained in Example 1 were placed on a sintered glass filter (G2) and a water jet vacuum was applied to the filter. The hydrogen peroxide released by the atmospheric pressure in the form of an aqueous solution was collected in a receiver. 33% of the hydrogen peroxide contained in the material used had been released within 15 min and a total of 50% within 60 min.

### Example 20

### Time-Delayed Release of Hydrogen Peroxide into a Pulverulent Medium

165 g of a commercial edible starch were mixed with 1.8 g of the product obtained in Example 1. The starch treated in this way was stored in an open container for 13 weeks at 20 to 23° C. and a humidity of 50 to 60%. No attack by mould or other microorganisms was detectable during storage. After storage, the treated starch displayed no deterioration in its free-flowing properties. After comparable storage, an untreated sample of the same starch displayed clumping and the first signs of mould infestation.

### Example 21

### Time-Delayed Release of Oxygen

The product obtained in Example 3 was stored at room temperature for a period of 60 days. During this time the hydrogen peroxide content of the powder fell from an initial 50.1 wt % to 45.6 wt % after 30 days and 40.5 wt % after 60 days. The decomposition of hydrogen peroxide during storage releases oxygen into the ambient air, whereby the amount of oxygen released is calculated at 15 Nl/kg after 30 days and 32 Nl/kg after 60 days (Nl=standard litres). Over a period of 60 days a uniform release of oxygen into the ambient air thus occurs at a rate of approx. 0.5 Nl/kg·d.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A pulverulent mixture comprising hydrogen peroxide and hydrophobized, pyrogenically prepared silicon dioxide powder, wherein the hydrophobized silicon dioxide powder has a methanol wettability of at least 40 and is present at less than 9 wt %, based on the total weight of the mixture, and the content of hydrogen peroxide, based on the total weight of the mixture, is between 10 and 50 wt %.

2. A process for producing the pulverulent mixture of claim 1, comprising treating a hydrophobized, pyrogenically prepared silicon dioxide powder having a methanol wettability of at least 40 with an aqueous hydrogen peroxide solution at a temperature of not more than 70° C.

3. The process of claim 2, wherein said silicon dioxide powder has been hydrophobized with octamethylcyclotetrasiloxane, polydimethylsiloxane, octylsilane and/or hexamethyl-disilazane.

4. The process of claim 3, wherein the specific surface area of the silicon dioxide powder is between 90 and 400 m²/g.

5. The process of claim 2, wherein the aqueous hydrogen peroxide solution has a content of hydrogen peroxide of between 5 and 70 wt %.

6. The process of claim 5, wherein the aqueous hydrogen peroxide solution is stabilized.

7. A composition comprising the pulverulent mixture of claim 1, wherein said composition is sleeted from the group consisting of: a) a topical medication for the treatment of acne; b) a detergent; c) a cleaning composition used in the absence of water; d) a skin treatment; e) a hair treatment; f) a hardener for curing a formulation comprising the pulverulent mixture as claimed in claim 1.

8. A method for the controlled release of hydrogen peroxide and/or oxygen, wherein hydrogen peroxide and/or oxygen is released from a pulverulent mixture comprising hydrogen peroxide and hydrophobized silicon dioxide.

9. The method of claim 8, wherein hydrogen peroxide is present in the pulverulent mixture in the form of drops of an aqueous solution of hydrogen peroxide enclosed by hydrophobized silicon dioxide.

10. The method of claim 8, wherein a pyrogenically produced, hydrophobized silicon dioxide having a methanol wettability of at least 40 is used as the hydrophobized silicon dioxide.

11. The method of claim 8, wherein the release of hydrogen peroxide and/or oxygen takes place in a time-delayed manner.

12. The method of claim 8, wherein hydrogen peroxide is released in response to the application of pressure.

13. The method of claim 8, wherein hydrogen peroxide is released into an aqueous medium.

14. The method of claim 13, wherein the pulverulent mixture is used in a container which is permeable for water and hydrogen peroxide and impermeable for the hydrophobized silicon dioxide.

15. The method of claim 8, wherein hydrogen peroxide is released into a pulverulent medium.

16. The method of claim 8, wherein the pulverulent mixture is added to an emulsion, a gel, a cream or a paste.

17. The method of claim 8, wherein hydrogen peroxide is released in an amount which inhibits the multiplication of microorganisms or in an effective amount to kill microorganisms.

18. The method of claim 8, wherein oxygen is released into a gaseous medium.

19. The method of claim 8, wherein oxygen is released in an amount which inhibits the formation of volatile metabolic products by anaerobic microorganisms.

20. The method of claim 8, wherein the pulverulent mixture is part of a wound treatment agent and wherein hydrogen peroxide is released during use of said wound treatment agent under the application of pressure in an amount which inhibits the multiplication of microorganisms.
